(19) United States
(12) Patent Application Publication
(10) Pub. No.: US 2010/0087796 A1
(43) Pub. Date: Apr. 8, 2010

(54) Method For Controlling A Pump Drive

(75) Inventor: George S. Baggs, Amherst, NY (US)

Correspondence Address: HODGSON RUSS LLP;THE GUARANTY BUILDING, 140 PEARL STREET, SUITE 100, BUFFALO, NY, 14202-4040, US

(73) Assignee: Reichert, Inc., Depew, NY (US)

(21) Appl. No.: 12/244,897

(22) Filed: Oct. 3, 2008

(51) Int. Cl. A61M 5/20; B67D 5/16

(52) U.S. Cl. 604/506; 604/151; 222/71

(57) ABSTRACT

A syringe pump and associated computer are provided to achieve an ultra-low flow rate. The computer may generate syntax commands to a motion controller of the syringe pump to displace a plunger by a selected number of steps or increments and pause a period of time before repeating the displace and pause steps. The cycle may be repeated until a desired end condition has been reached. The syringe pump and computer may be embodied as separate linked units or as a standalone unit wherein the computer is an embedded controller located within the syringe pump housing. The invention may also be embodied as a method for achieving ultra-low flow.

### FIELD OF THE INVENTION

The present invention relates in general to syringe pumps used to provide accurate and precise fluid flow. The present invention may be used to provide fluid flow of a liquid sample to a flow cell of an analytical instrument, for example a surface plasmon resonance (“SPR”) spectroscopy system. Other uses for the invention may include controlled delivery of drugs or nutritional solutions to a patient by way of a medical infusion pump.

### BACKGROUND OF THE INVENTION

Syringe pumps are used today in many applications including, for example flow cytometry, SPR analysis, chromatography, mass spectroscopy, microdialysis, and drug infusion. These applications require accurate and repeatable fluid flows in order to provide acceptable results.

For SPR analysis applications, infuse flow rates range from 0.1 to 3,000 μL/min, and refill flow rates range between 1,000 and 25,000 μL/min. Infuse flow rates lower than 5 μL/min are used during experiment setup, infuse flow rates between 25 and 100 μL/min are used during kinetics experiments, and infuse flow rates above 1,000 μL/min are used to clear out the system. The upper infuse flow rate is artificially set to prevent system over-pressure.

Syringe pumps exist which provide fluid flows on the order of 5-500 μL per minute and typically cost less than US $1,000. These syringe pumps are usually OEM modules, and not complete stand-alone syringe pumps. These modules contain a motion controller printed circuit board (PCB) which can execute pre-defined commands (e.g. start, stop, dispense or aspirate “x” steps at speed “y”, etc . . . ) from an external controller. An example of a syringe pump in this category is the Hamilton Company PSD/4. The minimum stepper motor speed for the PSD/4, which is dictated by the PSD/4's built-in motion controller, is 5 steps/sec. Therefore, the minimum possible flow rate achievable with the PSD/4 depends on the volume of the installed syringe. At 5 steps/sec, a minimum flow rate of 0.625 μL/min can be achieved with a 12.5 μL syringe; however, with a 500 μL syringe, the minimum flow rate is only 25 μL/min. As the syringe volume increases, so does the lowest possible flow rate.

During SPR kinetics experiments, experimental runs normally last between 5 and 10 minutes, but sometimes longer. Ideally, the experimental run can be completed before the PSD/4 syringe is emptied and a refill cycle is required because a refill event will upset the baseline in an SPR kinetics experiment. The maximum possible run time depends on the volume of the installed syringe. Only syringes with volumes of 500 μL or greater can provide long enough run times when infuse flow rates are between 25 and 100 μL/min. The problem is of course that these larger volume syringes do not allow the low flow rates needed during experimental setup due to the limitations of the PSD/4's built-in motion controller.

Pumps exist which provide ultra-low flow rates less than 5 μL/min using a larger volume syringe capable of supplying enough fluid to complete an experimental run without requiring a refill cycle, but these pumps cost much more than US $1,000. For example, syringe pumps made by Harvard Apparatus may cost US $5,000 or more. Such ultra-low flow syringe pumps are desirable for use in devices such as the SR7000 Surface Plasmon Resonance Refractometer made by Reichert, Inc., assignee of the present invention. However, the high cost of commercially available ultra-low flow syringe pumps can be prohibitive.

Accordingly, there is a need for a lower-cost syringe pump system which provides a wide range of fluid flow rates, including ultra-low flow rates, without limiting syringe volume, so that even lengthy SPR experimental runs may be completed without interruption for refill.

### BRIEF SUMMARY OF THE INVENTION

A syringe pump system formed in accordance with the present invention generally comprises a syringe pump, for example a Hamilton Company PSD/4, a motion controller that controls operation of a plunger drive of the syringe pump in accordance with syntax commands received by the motion controller, a computer that generates syntax commands based on desired flow parameters and transmits the syntax commands to the motion controller, and a storage medium having encoded thereon machine readable instructions executable by the computer to generate syntax commands causing the plunger drive to displace the plunger by at least one increment, pause for a specified time period, and repeat the displace and pause steps for the same or a different number of increments and the same or a different time period until an end condition has been reached. In this way, a flow rate less than the flow rate achieved by running the plunger drive at a minimum drive speed dictated by the motion controller can be realized. The end condition may be that the syringe is empty or the attached apparatus, if any, is full.

The computer may be a CPU provided as part of an external computer system, or a suitable embedded controller, such as a Z80® microcontroller as is commonly known in the industry, located within a housing of the syringe pump. In the former case, the storage medium may be a hard drive or other storage device associated with the external computer system. In the latter case, the storage medium may be on board memory provided as part of the embedded controller, and an input device and a display device may be disposed on the syringe pump housing so that a user may interact with the embedded controller to enter desired flow parameters and control pump operation.

The present invention may be embodied as a method for providing ultra-low flow from a syringe containing fluid, the syringe having a syringe body and a plunger displaceable relative to the syringe body to force fluid from the syringe. The method generally comprises the steps of a) displacing the plunger by at least one increment; b) pausing for a specified time period; and c) repeating the displace and pause steps for the same or a different number of increments and the same or different time periods until an end condition has been reached. The pause time period or duty cycle may be calibrated to achieve a desired average flow rate.

### DESCRIPTION OF THE DRAWINGS

For a fuller understanding of the nature and objects of the invention, reference should be made to the following detailed description taken in conjunction with the accompanying drawings, in which:

FIG. 1A is a perspective view of the front, top, and side of a prior art syringe pump;

FIG. 1B is a perspective view of the syringe pump of FIG. 1A showing the back of the syringe pump housing;

FIG. 2 is a block diagram of a syringe pump system formed in accordance with an embodiment of the present invention;

FIG. 3 is a perspective view of a syringe pump system formed in accordance with another embodiment of the present invention;

FIG. 4 is a block diagram of the syringe pump system shown in FIG. 3;

FIG. 5 is a plot of flow rate versus time for a syringe pump controlled to provide ultra-low flow in accordance with the present invention; and

FIG. 6 is a flow chart showing a method according to a further embodiment of the present invention.

### DETAILED DESCRIPTION OF THE INVENTION

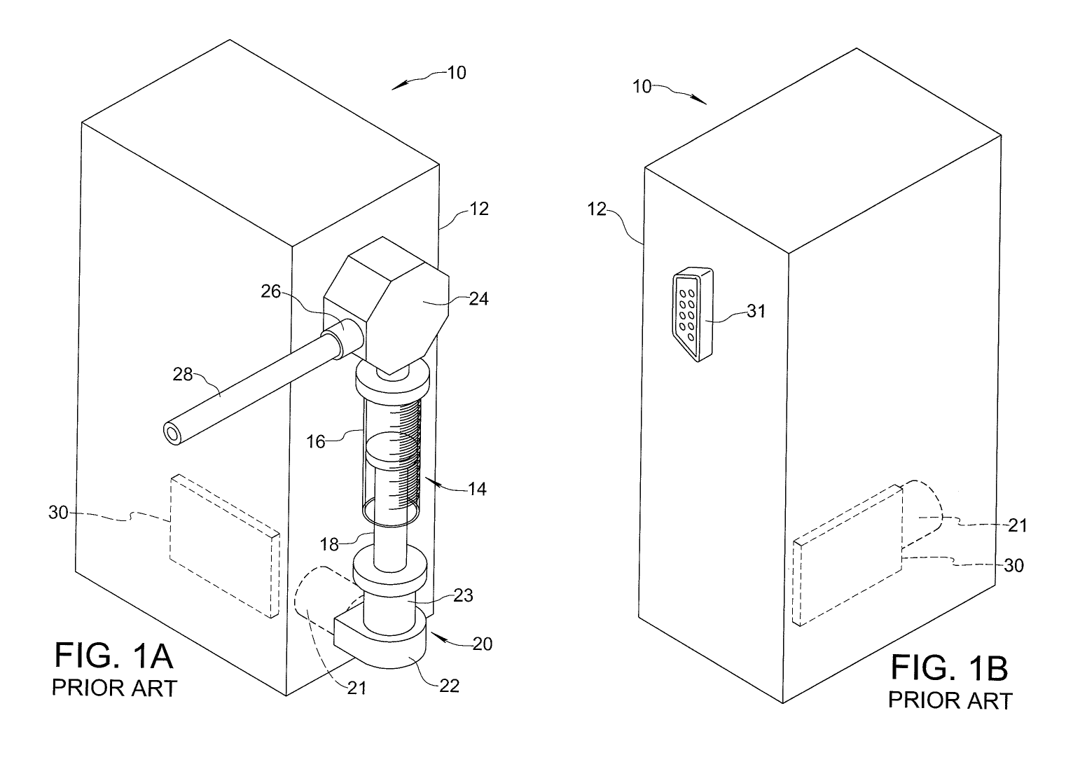

Reference is made initially to FIGS. 1A and 1B for description of a syringe pump 10 commonly known in the prior art. An example of a prior art syringe pump is the Hamilton Company PSD/4. Syringe pump 10 comprises a housing 12 and a syringe 14. The syringe 14 includes a syringe body 16 and a plunger 18 which can slide within the syringe body 16 to displace fluid that may be contained within the syringe body. Syringe 14 may be interchangeable, wherein syringes of different volumes each having a common stroke length of plunger 18, for example 30 mm, may be selected as needed for a given application. Plunger 18 is engaged by a plunger drive 20 which may cause the plunger 18 to move into and out of the syringe body 16 thereby forcing fluid into and out of syringe body 16. The depicted syringe pump 10 further comprises a valve assembly 24 which includes at least one fluid port 26. The fluid port 26 may be arranged in flow communication with an output tube 28 which in turn may be connected to a further device, for example a flow cell or an autosampler associated with an analytical instrument (not shown).

Plunger drive 20 includes a motor 21 (typically a D.C. stepper motor of a kind commonly known), an actuator 23 for drivably engaging plunger 18, and a drive assembly 22 which mechanically connects motor 21 to actuator 23 such that rotational motion of the motor is transmitted to the plunger as linear motion.

Typically, and as is the case with the PSD/4, housing 12 also encloses a motion controller 30 that controls operation of plunger drive 20 in accordance with syntax commands received by the motion controller 30. FIG. 1B shows the back of syringe pump 10 on which may be located a communication port 31 of a kind commonly known in the art—for example, RS-232, USB, etc., whereby syntax commands may be inputted to motion controller 30. The motion controller 30 converts the received syntax commands into motor drive signals for controlling operation of plunger drive 20 in accordance with the syntax commands. For example, the syntax command “ZR” results in generation of a drive signal that causes plunger drive 20 to return to a “home” position and set the valve assembly 24 (which may also be automated) to the correct fluid port 26.

Prior art syringe pumps such as the PSD/4 are controlled with a number of initial parameters, such as desired precision (the motor may be able to selectively move the plunger drive in increments or “steps,” in the case of the PSD/4, the precision is selectable between increments of 1/3000^{th} or 1/24,000^{th} of the syringe volume) or a desired velocity (the syringe pump may, for example, dispense the entirety of the syringe in a time selected from between 1 second to 20 minutes). Following the setting of initial parameters, the syringe pump may then be controlled with an “action command” to move the plunger by a desired number of steps (increments or incremental movements) in order to dispense a desired volume of fluid. The steps or incremental movements are carried out one after another by the stepper motor until the desired number of steps is reached. As mentioned above, motion controller 30 defines a minimum drive speed for plunger displacement. For example, the minimum stepper motor speed of the PSD/4 dictated by motion controller 30 is 5 steps/sec.

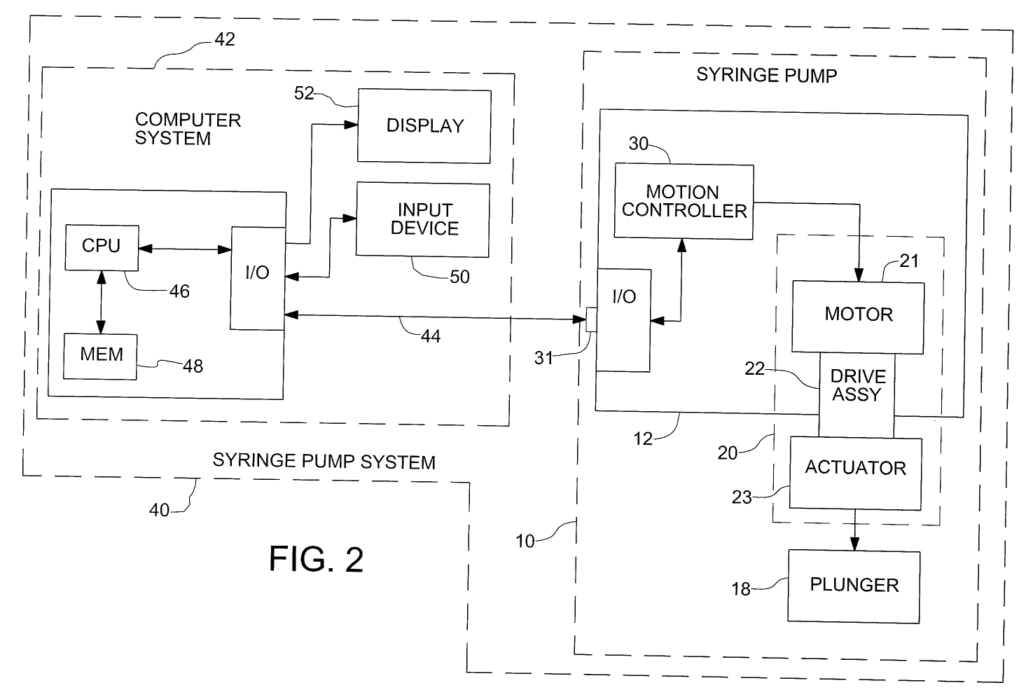

FIG. 2 depicts a syringe pump system 40 embodying the present invention. Syringe pump system 40 includes syringe pump 10 and a computer system 42 connected to syringe pump 10 by a cable 44 connected to communications port 31 or by a wireless communication system. Computer system 42 includes a central processing unit (CPU) 46 and a machine readable storage medium 48 connected to the CPU, such as a hard drive, floppy drive, memory chip, or the like, which stores machine-readable instructions executable by CPU 46 and may also store data describing physical characteristics of pump system 40. In the present embodiment, CPU 46 may act as an external controller, wherein “external” is intended to mean that it is outside of syringe pump housing 12. CPU executes the machine readable instructions to generate syntax commands based on desired flow parameters entered by a user, and transmits the syntax commands to motion controller 30 for operating plunger drive 20 to achieve the desired flow parameters. Motion controller 30 energizes stepper motor 21 in accordance with the syntax commands, and the incremental steps of the stepper motor are conveyed by drive assembly 22 to actuator 23 engaging an end of plunger 18. Computer system 42 further includes an input device 50, such as a keyboard, mouse, touch pad, trackball, etc., and a display 52, thereby enabling a user to interact with CPU 46 and storage medium 48. Thus, the user may enter desired flow parameters and initiate a controlled plunger stroke by way of computer system 42.

In accordance with the present invention, the machine readable instructions executed by CPU 46 may provide for a delay or pause syntax command between instances of the action syntax command. As an example, computer system 42 may send a series of syntax commands to motion controller 30 to move the plunger 18 by one step (one increment), then pause the plunger for a time period of 250 ms, followed by another displacement increment, and another pause, etc. This sequence may be repeated until a desired end condition is met, for example, a particular fluid volume is discharged, plunger displacement is achieved, or time period has elapsed. In this way, the syringe pump 10 may be caused to dispense fluid at a flow rate slower than that corresponding to the slowest speed of stepper motor 21 dictated by motion controller 30.

Table 1 shows normal flow rates achievable by the PSD/4 (“normal flow rate”) by running the stepper motor at its predefined minimum and maximum drive speeds dictated by motion controller 30, along with ultra-low flow rates achievable by inserting syntax commends for pausing the plunger drive between displacement syntax commands according to the inventive system. It should be noted that the maximum normal flow rate is always artificially limited to prevent overpressure/damage to the valve assembly.

TABLE 1

| | Minimum | | | |
|---|---|---|---|---|
| Syringe | normal flow | Maximum | Ultra-low | |
| Volume | rate | normal flow rate | flow rate | Delay time |
| 1000 μL | 50 μL/min | 500 μL/min | 1.0 μL/min | 2500 ms |
| | | | 2.5 μL/min | 1000 ms |
| | | | 5.0 μL/min | 500 ms |
| 500 μL | 25 μL/min | 500 μL/min | 1.0 μL/min | 1250 ms |
| | | | 2.5 μL/min | 500 ms |
| | | | 5.0 μL/min | 250 ms |
| 100 μL | 5 μL/min | 500 μL/min | 1.0 μL/min | 250 ms |
| | | | 2.5 μL/min | 100 ms |

It will be apparent that other ultra-low flow rate values will be achievable by varying the delay time (the pause time period) and/or the number of displacement steps between pauses. The delay time period may be altered by issuing successive pause syntax commands of the same duration, or by issuing a pause syntax command having a different time period parameter. Conceptually, the “duty cycle” of the flow pulses may be varied (2 pauses and 1 step equals a 33% duty cycle; 1 pause and 1 step equals a 50% duty cycle; 1 pause and 3 steps equals a 75% duty cycle; and so on). By varying the duty cycle during a plunger stroke, the flow rate may be changed as a function of time to provide a “gradient” flow rate. For example, computer system 42 may command motion controller 30 to displace plunger 18 by one increment followed by a 250 ms delay and repeat this for 200 cycles. Then, the delay time may be changed, for example, increased to 500 ms for an additional 200 cycles. In another example, the delay time may be increased by 25 ms per step until it reaches 500 ms. It is also contemplated construct a drive cycle having a single pause syntax command after a series of successive displacement syntax commands.

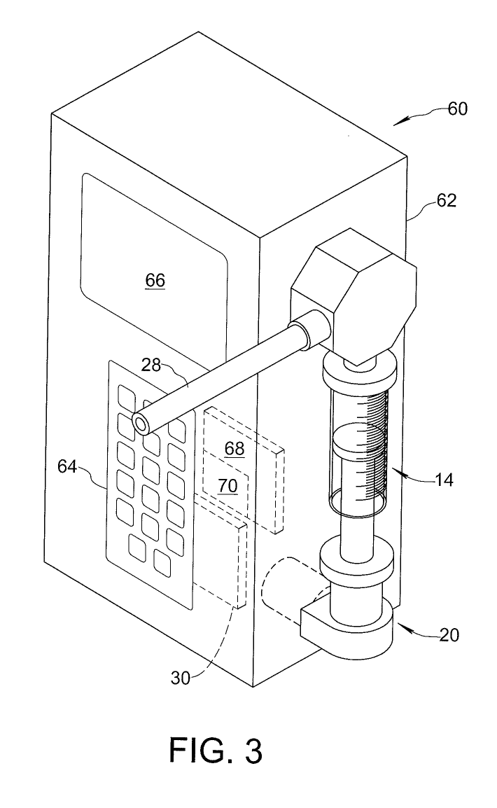

Ultra-low flow rates are generated by averaging flow pulses as shown in FIG. 3. The actual flow versus time profile appears like the humps, and the delay (pause) time period is calculated to yield an average flow rate matching a desired flow rate setting. Experiments with a device according to the invention have shown that displacement-pause cycles of the magnitudes shown in Table 1 above do not create unacceptable variations in the flow exiting syringe pump 10. Empirical testing has revealed that the average ultra-low flow rate can be calibrated to within ±1.0% of the desired flow rate set by a user. It is also contemplated to provide a flow accumulator (not shown) in the syringe pump system to mitigate flow pulsations.

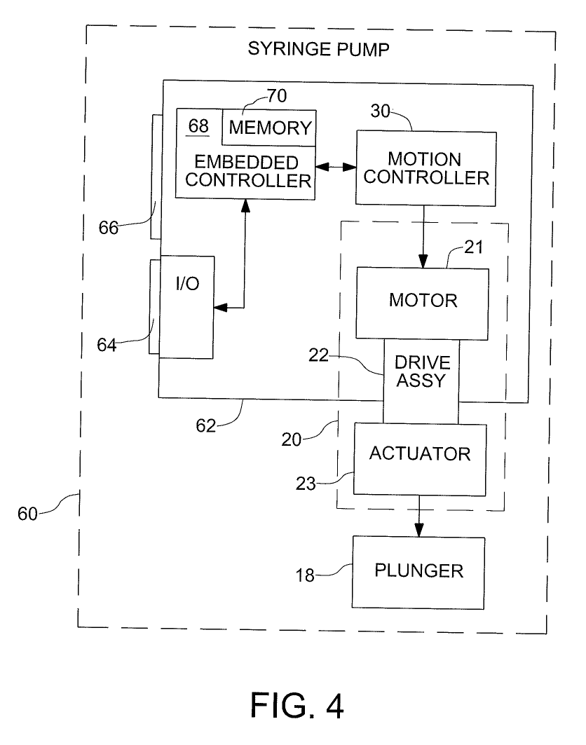

In another embodiment of the present invention, shown in FIG. 4, a syringe pump system 60 is shown. A computer in the form of an embedded digital controller 68 may be installed within the syringe pump housing 62. An example of the type of embedded controller that could be mounted in such a fashion is a Rabbit® Semiconductor RCM3700 RabbitCore™, or other small PCB controller commonly known. Controller 68 is connected to a memory device 70 storing machine readable instructions as described above. Memory 70 may be “on board memory” provided directly on embedded controller 68 as shown in FIG. 3, or a physically separate memory device within housing 62. As will be appreciated, syringe pump system 60 is “self-contained” such that it may be installed in a rack or otherwise used without attachment to an external controller. The syringe pump system 60 of this embodiment may further contain communication ports for connection of one or more input devices and/or display devices for enabling a user to enter desired flow parameters to embedded controller 68 and control pump operation. Such communication ports are commonly known, such as, for example, USB, RS232, RS485, and Ethernet ports, and the like. Alternatively, or additionally, an input keypad 64 and/or a display 66 may be integrated with the housing 62 and connected to embedded controller 68 for enabling a user to enter desired flow parameters and control pump operation. Such integration may be in any convenient location on the housing such as a side panel, front panel, or top panel. Embedded controller 68 is programmed to generate syntax commands based on entered flow parameters and issue the syntax commands to motion controller 30 to control operation of plunger drive 20. In accordance with the present invention, the machine readable instructions stored in memory 70 and executed by embedded controller 68 may provide for a delay or pause syntax command between instances of the action syntax command.

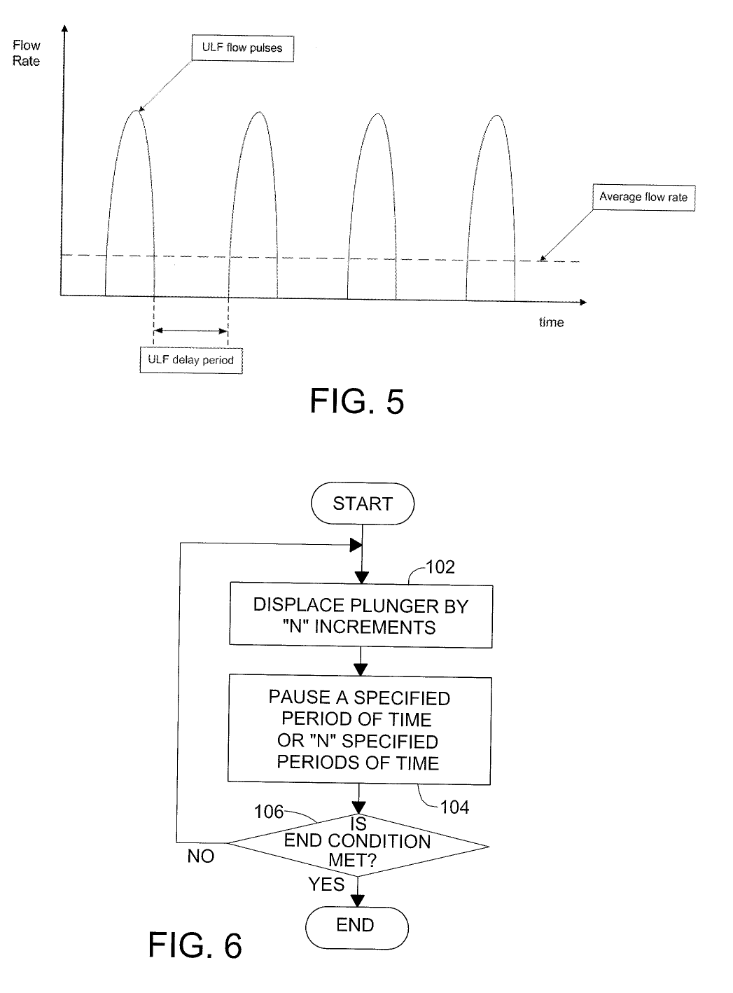

The present invention may be embodied as a method, depicted in FIG. 5, for providing flow at an ultra-low flow rate. The method comprises step 102 of displacing a plunger of the syringe by at least one increment, which may correspond to a revolution step of stepper motor 21. This is followed by step 104 of pausing plunger displacement for a specified period of time. A logic step 106 occurs next, namely determining whether a particular end condition has been met, for example, whether a desired amount of fluid has been displaced. If not, procedural flow returns to step 102, and steps 102 and 104 are repeated. If the end condition has been met, then the method terminates.

Although the present invention has been described with respect to various particular embodiments, it will be understood that other embodiments of the present invention may be made without departing from the spirit and scope of the present invention. Hence, the present invention is deemed limited only by the appended claims and the reasonable interpretation thereof.

What is claimed is:

1. A syringe pump system comprising:
a) a syringe pump including a syringe and a plunger drive, wherein the syringe has a syringe body and a plunger received by the syringe body, and the plunger drive is operable to displace the plunger relative to the syringe body;
b) a motion controller that controls operation of the plunger drive in accordance with syntax commands received by the motion controller, the motion controller defining a minimum drive speed for plunger displacement;
c) a computer that generates syntax commands based on desired flow parameters and transmits the syntax commands to the motion controller; and
d) a machine readable storage medium connected to the computer, the storage medium having encoded thereon machine readable instructions executable by the computer to generate syntax commands causing the plunger drive to:
i. displace the plunger by at least one increment;
ii. pause for a specified time period; and
iii. repeat the displace and pause steps for the same or a different number of increments and the same or a different time period until an end condition has been reached;
wherein a flow rate less than a flow rate corresponding to the minimum drive speed can be achieved.

2. The syringe pump system of claim 1, wherein the syringe pump further includes a syringe pump housing on which the syringe is mounted, and the computer and storage medium are external to the syringe pump housing.

3. The syringe pump system of claim 1, wherein the syringe pump further includes a syringe pump housing on which the syringe is mounted, and the computer and storage medium are internal to the syringe pump housing.

4. The ultra-low flow pump system of claim 3, further comprising an input device and a display each mounted on the syringe pump housing and connected to the internal computer.

5. The syringe pump system of claim 1, wherein the machine readable instructions cause the syringe pump to have a flow rate ranging from 1-5 μL/min when the instructions are executed by the computer.

6. The syringe pump system of claim 1, wherein the end condition is met when a specified volume of fluid has been discharged from the syringe.

7. The syringe pump system of claim 1, wherein the end condition is met when a specified amount of time has elapsed relative to a start time.

8. The syringe pump system of claim 1, wherein the end condition is met when the plunger has been displaced by a specified amount from a start position.

9. The syringe pump system of claim 1, wherein the pause time period provided by the machine readable instructions remains constant.

10. The syringe pump system of claim 1, wherein the pause time period provided by the machine readable instructions changes.

11. The syringe pump system of claim 1, wherein the number of displacement increments provided by the machine readable instructions remains constant.

12. The syringe pump system of claim 13, wherein the number of displacement increments provided by the machine readable instructions is one.

13. The syringe pump system of claim 1, wherein the number of displacement increments provided by the machine readable instructions changes.

14. In a syringe pump system having a stepper motor operable for displacing a syringe plunger relative to a syringe body; a motion controller that controls operation of the stepper motor in accordance with syntax commands received by the motion controller, the motion controller defining a minimum drive speed for plunger displacement; and a computer that generates syntax commands based on desired flow parameters and transmits the syntax commands to the motion controller; the improvement comprising:
a machine readable storage medium connected to the computer, the storage medium having encoded thereon machine readable instructions executable by the computer to generate syntax commands, wherein the machine readable instructions include instructions for pausing actuation of the stepper motor for a specified period of time between separate syntax commands actuating the stepper motor to provide a flow rate less than a flow rate corresponding to the minimum drive speed.

15. The improvement according to claim 16, wherein the specified period of time is in a range from 100 milliseconds through 2500 milliseconds.

16. A method for reducing the flow rate of flow from a syringe containing fluid, the syringe having a syringe body and a plunger displaceable by a stepper motor relative to the syringe body to force fluid from the syringe, the method comprising the steps of:
a) actuating the stepper motor through at least one increment to displace the plunger;
b) pausing the stepper motor for a specified time period; and
c) repeating the actuating and pausing steps for the same or a different number of increments of the stepper motor and the same or different time periods until an end condition has been reached.

17. The method according to claim 18, wherein the specified time period is always in a range from 100 milliseconds through 2500 milliseconds and the at least one stepper motor increment is always one increment.
